(19) United States
(12) Patent Application Publication
(10) Pub. No.: US 2007/0189545 A1
(43) Pub. Date: Aug. 16, 2007

(54) Audiometer

(75) Inventors: Wolfgang Geiger, Lehrberg (DE); Uwe Rass, Nurnberg (DE); Robert Ryman, Bucks (GB)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AUDIOLOGISCHE TECHNIK GMBH

(21) Appl. No.: 11/699,674

(22) Filed: Jan. 30, 2007

(30) Foreign Application Priority Data
Jan. 30, 2006 (DE) 10 2006 004 362.6

(51) Int. Cl. H04R 29/00; A61B 5/00

(52) U.S. Cl. 381/60; 600/559; 73/585

(57) ABSTRACT

The aim is to simplify resource planning in a clinic or by a hearing aid technician. For this purpose, an audiometer and a method for operating an audiometer are proposed in which, following a simple hearing test, in addition to information as to whether treatment of a user with a hearing aid is necessary, the presence of any hearing-loss class in which the user can be grouped is also shown. In this way, the expense with regard to time, materials and personnel required for matching a hearing aid to a particular user can be more easily estimated.

### CROSS REFERENCE TO RELATED APPLICATIONS

This application claims priority of German application No. 10 2006 004 362.6 filed Jan. 30, 2006, which is incorporated by reference herein in its entirety.

### FIELD OF THE INVENTION

The invention relates to an audiometer with means of stimulating the hearing of a user and means for detecting a reaction of the user to the stimulation. The invention also relates to a method for operating an audiometer and to a hearing aid.

### BACKGROUND OF THE INVENTION

Before a user is provided with a hearing aid, the hearing loss of the user is first determined by using a suitable audiometer. To do this, the hearing is stimulated by test signals at different signal frequencies and different sound pressure levels and the reaction of the user to a particular test signal is detected. Based on the results of the hearing test, a hearing-aid technician selects a suitable hearing aid to compensate for the particular hearing loss. The signal transmission characteristic of modem hearing aids can be matched to the particular hearing loss of the user by programming.

From EP 1 073 314 A1, a method is known for adjusting a hearing aid whereby a user is stimulated by different test signals, the reactions of the user to the test signals are detected and hearing aid-specific adjustment parameters for the hearing aid are generated and transmitted to the hearing aid. A method of this kind and an associated test and adapter unit are known from U.S. Pat. No. 5,197,332.

From DE 88 03 680 U1, an audiometer is known that has fully automatic user guidance, with an audiogram and a short description of the test results, perhaps as “impaired hearing in left ear” being printed by a computer at the end of the hearing test.

Different categories of hearing aid that are more or less suitable for the treatment of a specific user with respect to particular hearing loss are available to treat a hearing aid user. Examples of different categories of hearing aid are in-ear hearing aids, behind-ear hearing aids for an open treatment or behind-ear hearing aids with a customized otoplasty (closed treatment). Up to now it has been the task of the hearing aid technician to choose a hearing aid category for the user. The time required for this process of matching hearing aids from different categories to a user varies substantially.

### SUMMARY OF THE INVENTION

The object of the invention is to simplify the resource planning required in a clinic or by a hearing aid technician.

The object is achieved by an audiometer and by a method as claimed in the claims.

The audiometer according to the invention has means of stimulating the hearing of a user. This can be a simple sound generator by means of which the user is exposed to test signals of different frequencies and sound levels through a loudspeaker or headset. The hearing of the user can also be stimulated by other means such as structure-borne sound.

The audiometer according to the invention also has means for detecting at least one reaction of the user to the particular stimulation. This can, for example, be a push switch that is pressed by the user immediately he hears a sound. Other reactions of the user, for example involuntary reactions of the body such as otoacoustic emissions, can however also be detected. The detected reactions are then automatically evaluated in such a way that the result of the evaluation of the individual hearing loss of the user can be assigned to a specific hearing-loss class from a number of possible hearing-loss classes.

A simple means of classifying hearing loss through different hearing-loss classes is by characterizing the hearing loss in different grades (e.g. “slight”, “moderate” or “severe”) in different frequency ranges (e.g. low-tone range, medium tone range, high-tone range). A specific hearing-loss class can thus be determined by a moderate loss of hearing in the low-tone range, a slight hearing loss in the medium frequency range and a severe hearing loss in the high-tone range. Other differentiating features of different hearing-loss classes are however also possible. For example the class of hearing loss that can be suitably treated only by a hearing aid worn behind the ear with a customized ear adapter piece, or the hearing-loss class a characteristic of which is that an open treatment to compensate for the hearing loss is also suitable.

The audiometer according to the invention advantageously includes a visual display by means of which the result of the hearing test is shown to the user. For example, the illumination of a red light means that a closed treatment with a customized ear piece is required. If, on the other hand, an orange-colored light illuminates, this means that an open treatment is also suitable. A green light can, for example, indicate that there is no significant hearing loss present and no hearing aid is necessary. Naturally, there are many other possibilities in addition to the indications already mentioned to indicate the results of hearing tests by visual (e.g. by means of a display), acoustic (e.g. by means of voice output) or tactile means.

The invention has the advantage that by means of a quick and simple test the user is not only shown whether treatment with a hearing aid is recommended but also the hearing-loss class to which the user can be assigned is indicated, so that the type of treatment (hearing aid category) suitable for the user is determined at the same time. The cost in terms of time, personnel and money required to match a hearing aid to this user can then be estimated from this. This simplifies the workflow when matching the hearing aid.

The invention is particularly advantageous if during hearing screening, i.e. screening testing, a large number of persons are to be tested within a short time. The group of persons for whom no hearing aid treatment is necessary can thus quickly be distinguished from the group of persons with reduced hearing. For the group of persons with reduced hearing, the relevant hearing aid category for the particular person is then determined and therefore the costs in term of time, material and personnel required for the care and treatment of the particular person with a hearing aid can be more easily calculated.

A development of the invention provides that data generated by the audiometer can be transmitted to a hearing aid adapter unit or directly to a hearing aid. The audiometer has a suitable interface for this purpose. However, the data is preferably transmitted wirelessly with suitable transmitting and/or receiving means for wireless signal transmission being present at the audiometer.

The individual results of the respective hearing test that has been carried out, data for identification of the determined hearing-loss class or also data resulting from this, for example hearing aid parameters for adapting the signal processing in the hearing aid to the determined hearing loss, can be transmitted.

Where there is direct data transmission to a hearing aid, the relevant hearing aid can thus be adjusted without an adapter unit and without adaptation software, which is useful for areas with a reduced infrastructure.

Where the data is transmitted to an adapter unit, a suitable adaptation strategy and/or suitable adaptation parameters can be preset. More complicated settings and optimizations can be carried out later by a technically skilled person.

### BRIEF DESCRIPTION OF THE DRAWINGS

The invention is explained in more detail in the following with the aid of an exemplary embodiment. The drawings are as follows.

FIG. 1 An audiometer according to the invention,

FIG. 2 The flowchart of a hearing test carried out with the audiometer.

### DETAILED DESCRIPTION OF THE INVENTION

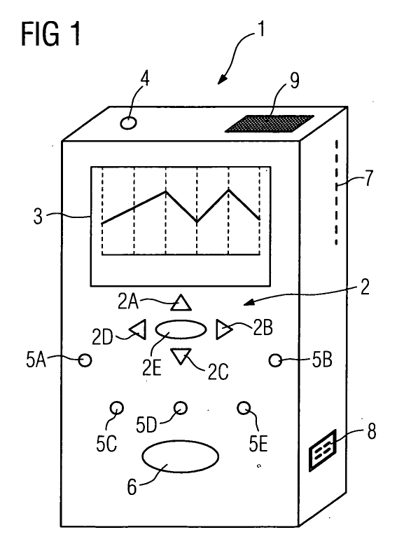

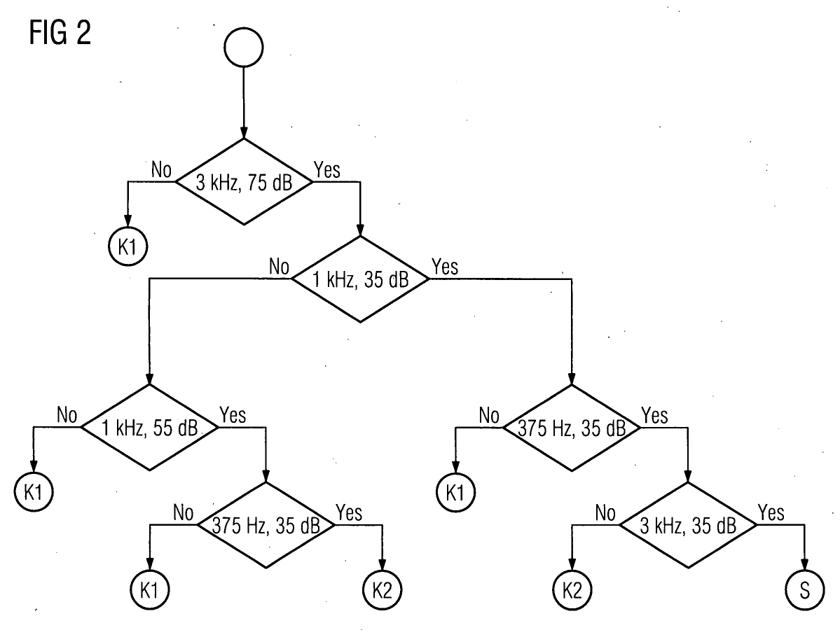

The audiometer 1 shown in FIG. 1 shows a control device 2 with arrow keys 2A, 2B, 2C and 2D and a control element 2E arranged centrally between the arrow keys. A function of the control device 2 is to select and start a simple hearing test in conjunction with a display 3. During this hearing test, the user is exposed to different frequencies and volumes through a headset (not illustrated). In this case, the headset is connected to a connecting socket 4 on the audiometer 1. A first LED 5A illuminates immediately the audiometer 1 is ready for operation. A second LED 5B signals that a test tone is being output. The test tone is output for only a short duration, e.g. 3 seconds. If the user can hear the output test tone, he confirms this immediately by pressing push switch 6. If he cannot hear the test tone, there is then no operation of the push switch 6 or other control. After a short pause has elapsed after the output of the first test tone, a second test tone at a different frequency and/or volume to the first test tone is output. A flow chart of a possible sequence of test tones, dealt with in more detail at a later point, is shown in FIG. 2. It is pointed out that in the context of the invention a number of different sequences of tones, noises or test signals in general is possible that are, however, matched to each other in such a way that from the reactions of the user a specific hearing-loss class can be determined and therefore also the possibility is presented of being able to appropriately treat the specific hearing loss of the user with a hearing aid of a particular hearing aid category. For the audiometer I according to the exemplary embodiment, a red LED 5C, an orange LED 5D and a green LED 5E are present. If at the end of the hearing test, the red LED 5C illuminates, this means that only treatment with a hearing aid with a customized otoplasty is possible. If the orange LED 5D illuminates, this indicates that an open treatment is also possible. If the green LED 5E illuminates, this indicates that no hearing aid is necessary.

By means of the invention, the type of hearing aid a particular user requires can be quickly and easily determined, which also enables the time required to match a corresponding hearing aid to be approximately estimated.

The invention is then particularly advantageous where a number of persons are to be examined within a short period and treated with a hearing aid as necessary. Up to now, the time required for hearing aid adaptation for a person with hearing loss was very difficult to estimate during such hearing screening. With the preferred form of embodiment of an audiometer according to the invention, there is the possibility of data transmission to other devices, e.g. a PC (not illustrated). This is indicated on audiometer 1 by an antenna 7 and a socket 8 that enable a wireless connection or a cable connection to other devices. In this way, the data obtained from individual persons can be transmitted to a PC and further processed there. This enables better planning of the required resources. It is even possible to allow the data to feed into an electronically controlled workflow management system, such as is, for example, provided in some clinics. Resource planning can then be largely automated.

It is also possible to transmit the data generated with the audiometer 1 to a hearing aid adapter unit or directly to a hearing aid. In this case, the data can, for example, be used for an initial adaptation of a hearing aid (first fit). Furthermore, it is possible to transmit hearing aid adaptation parameters generated in the audiometer using the generated data to a relevant hearing aid for adaptation of the signal processing in the hearing aid to the particular hearing loss of a user. A further hearing aid adapter unit is then unnecessary.

The audiometer 1 according to the exemplary embodiment is advantageously provided with a display 3 by means of which, the hearing loss of the user can be graphically displayed relative to frequency. Furthermore, the display 3 can also be used to indicate the result of a hearing test according to the invention. This is then particularly advantageous if a very fine classification of possible hearing losses into a number of hearing aid classes is being made. Equally, a very much finer classification of hearing aids into different categories is possible. Just with in-ear devices, for example, a distinction can be made between CIC (completely in the canal), ITC (in the canal) and concha devices, in each case with a universal-fit housing shell or a custom-fit housing. An indication by means of LEDs, as in the exemplary embodiment, then becomes unclear. Furthermore, it is frequently possible that a specific hearing loss can be suitably treated using hearing aids of different categories. This circumstance may also be better indicated by an indication on a display.

The audiometer 1 according to the exemplary embodiment also has a loudspeaker 9 through which test signals can also be output or the result of a hearing test acoustically indicated.

There is a series of alternatives available for the external design of an audiometer according to the invention. For example, the audiometer as shown in FIG. 1 can be a hand-held unit. A larger design, e.g. in the form of a desk-top device, could also be used. A multipart design, e.g. with a separate display device (screen) is also possible.

The audiometer according to the invention is, however, advantageously structurally combined with devices with another function or integrated into these devices. An audiometer can thus, for example, also be designed as a remote control for a hearing aid, even in fact as a hearing aid itself. The integration of the functionality of the hearing aid according to the invention into a commercial audiometer is also conceivable. Furthermore, the audiometer according to the invention can also be integrated into a complex computer system, e.g. a workflow management system for a clinic.

FIG. 2 shows a flow diagram of an example of a hearing test according to the invention. The sequence proceeds in the following steps:
Stimulation of the user with a first acoustic signal with a first signal frequency and a first sound pressure level,
Indication of a first hearing aid category K1 by the audiometer in a case where the first acoustic signal was not heard by the user, and ending of the process,
Stimulation of the user with a second acoustic signal with a second signal frequency and a second sound pressure level in a case where the first signal was heard by the user,
Stimulation of the user with a third acoustic signal with a second signal frequency and a third sound pressure level in a case where the second acoustic signal was not heard by the user,
Indication of the first hearing aid category K1 by the audiometer in a case where the third acoustic signal was not heard by the user, and ending of the process,
Stimulation of the user with a fourth acoustic signal with a third signal frequency and a second sound pressure level in a case where the third acoustic signal was heard by the user,
Indication of the first hearing aid category K1 by the audiometer in a case where the fourth acoustic signal was not heard by the user, and ending of the process,
Indication of a second hearing aid category K2 by the audiometer in a case where the fourth acoustic signal was heard by the user, and ending of the process,
Stimulation of the user with a fifth acoustic signal with a third signal frequency and a second sound pressure level in a case where the second acoustic signal was heard by the user,
Indication of a first hearing aid category K1 by the audiometer in a case where the fifth acoustic signal was not heard by the user, and ending of the process,
Stimulation of the user with a sixth acoustic signal with a first signal frequency and a second sound pressure level in a case where the fifth acoustic signal was heard by the user,
Indication of a second hearing aid category K2 by the audiometer in a case where the sixth acoustic signal was not heard by the user, and ending of the process,
Indication of a symbol S by the audiometer in a case where the sixth acoustic signal was heard by the user, and ending of the process. The symbol S indicates that there is no hearing loss present that requires treatment.

With the audiometer according to the exemplary embodiment shown in FIG. 1, a red LED signals that severe hearing loss is present and treatment with a hearing aid of a first category, for example an HdO hearing aid with a customized otoplasty, is necessary. An orange LED signals that slight to moderate hearing loss is present that can also be suitably dealt with by open treatment.

For example, for the first signal frequency 3 kHz can be used, for the second signal frequency 1 kHz and for the third signal frequency 375 Hz can be used. Furthermore, for the first sound pressure level, for example, 75 dB can be used, 35 dB for the second sound pressure level and 55 dB for the third sound pressure level.

It is again pointed out that both the given numerical values and also the choice and sequence of test signals and test steps can differ considerably from the exemplary embodiment without this departing from the protective area of the invention.

1.-18. (canceled)

19. An audiometer for detecting a hearing loss of a person, comprising:
a stimulation device that stimulates a hearing of the person;
a detection device that detects a reaction of the person to the stimulation; and
an evaluation device that automatically evaluates the detected reaction so that a specific hearing loss class defining a degree of hearing difficulty is assigned to the person.

20. The audiometer as claimed in claim 19, wherein the hearing loss class is selected from a plurality of possible hearing loss classes.

21. The audiometer as claimed in claim 19, further comprising a device that indicates the assigned hearing loss class.

22. The audiometer as claimed in claim 21, further comprising a device that indicates a specific hearing aid category with a suitable treatment for the specific hearing loss class assigned to the person.

23. The audiometer as claimed in claim 22, wherein the hearing loss class or the hearing aid category is visually indicated.

24. The audiometer as claimed in claim 22, wherein the hearing aid category is selected from a plurality of possible hearing aid categories.

25. The audiometer as claimed in claim 19, wherein the stimulation device is a sound generator.

26. The audiometer as claimed in claim 25, wherein a plurality of acoustic signals with a plurality of different signal frequencies is generated by the sound generator and is outputted for stimulating the hearing.

27. The audiometer as claimed in claim 25, wherein a plurality of acoustic signals with a plurality of different sound pressure levels is generated by the sound generator and is outputted for stimulating the hearing.

28. The audiometer as claimed in claim 19, wherein data of the hearing loss class is transmitted to a hearing aid or a hearing aid adapter unit.

29. The audiometer as claimed in claim 28, wherein a hearing aid setting parameter is generated from the data of the hearing loss class and is transmitted to the hearing aid.

30. The audiometer as claimed in claim 19, wherein the audiometer is a remote control for a hearing aid.

31. The audiometer as claimed in claim 19, wherein the audiometer is integrated into a computer-aided workflow management system.

32. A hearing aid, comprising:
an audiometer integrated with the hearing aid that detects a hearing loss of a user of the hearing aid, the audiometer comprising:
a sound generator that stimulates a hearing of the person,
a detection device that detects a reaction of the person to the stimulation, and
an evaluation device that automatically evaluates the detected reaction so that a specific hearing loss class defining a degree of hearing difficulty is assigned to the person.

33. A method for detecting a hearing loss of a person using an audiometer, comprising:
stimulating a hearing of the person with a plurality of different acoustic signals;
detecting reactions of the person to the different acoustic signals;
evaluating the reactions;
determining a hearing loss class of a degree of hearing difficulty to the person based on the evaluation; and
outputting the determined hearing loss class.

34. The method as claimed in claim 33, wherein the different acoustic signals comprise a plurality of different signal frequencies or a plurality of different sound pressure levels.

35. The method as claimed in claim 33, wherein a hearing aid category is determined based on the hearing loss class with a hearing aid treatment suitable for compensating the hearing loss of the person and the determined hearing aid category is outputted.

36. The method as claimed in claim 33, further comprising:
stimulating the hearing of the person with a first acoustic signal with a first signal frequency and a first sound pressure level,
indicating a first hearing aid category by the audiometer if the person does not hear the first acoustic signal and ending the stimulating,
stimulating the hearing of the person with a second acoustic signal with a second signal frequency and a second sound pressure level if the person hears the first acoustic signal,
stimulating the hearing of the person with a third acoustic signal with a second signal frequency and a third sound pressure level if the person does not hear the second acoustic signal,
indicating a first hearing aid category by the audiometer if the person does not hear the third acoustic signal and ending the stimulating,
stimulating the hearing of the person with a fourth acoustic signal with a third signal frequency and a second sound pressure level if the person hears the third acoustic signal,
indicating a first hearing aid category by the audiometer if the person does not hear the fourth acoustic signal and ending the stimulating,
indicating a second hearing aid category by the audiometer if the person hears the fourth acoustic signal and ending the stimulating,
stimulating the hearing of the person with a fifth acoustic signal with a third signal frequency and a second sound pressure level if the person hears the second acoustic signal,
indicating a first hearing aid category by the audiometer if the person does not hear the fifth acoustic signal and ending the stimulating,
stimulating the hearing of the person with a sixth acoustic signal with a first signal frequency and a second sound pressure level if the person hears the fifth acoustic signal,
indicating a second hearing aid category by the audiometer if the person does not hear the sixth acoustic signal and ending the stimulating, and
displaying a symbol by the audiometer to indicate that the person hears the sixth acoustic signal and ending the stimulating.

37. The method as claimed in claim 36, wherein the first signal frequency is 3 kHz, the second signal frequency is 1 kHz, and the third signal frequency is 375 Hz.

38. The method as claimed in claim 36, wherein the first sound pressure level is 75 dB, the second sound pressure level is 35 dB, and the third sound pressure level is 55 dB.
